(19) United States
(12) Patent Application Publication
(10) Pub. No.: US 2021/0085623 A1
(43) Pub. Date: Mar. 25, 2021

(54) Formulation

(71) Applicant: Medherant Limited, Conventry (GB)

(72) Inventor: David Haddleton, Coventry (GB)

(73) Assignee: Medherant Limited, Conventry (GB)

(21) Appl. No.: 16/610,193

(22) PCT Filed: Apr. 30, 2018
(86) PCT No.: PCT/GB2018/051142

§ 371 Date: Nov. 1, 2019

(30) Foreign Application Priority Data
May 2, 2017 (GB) 1706969.1

(51) Int. Cl. A61K 9/70; A61K 31/192; A61K 31/167; A61K 47/10; A61K 47/14; A61K 47/20; A61K 47/34

(52) U.S. Cl. CPC A61K 9/7023; A61K 31/192; A61K 31/167; A61K 47/34; A61K 47/14; A61K 47/20; A61K 47/10

(57) ABSTRACT

The invention relates to transdermal drug delivery patches, in particular the provision of excipients that augment the drug release properties of the patches. The excipients typically involve a mixture of glycols, alkoxy alcohol and sulfoxides depending on the particular drug to be delivered.

### FIELD OF INVENTION

The invention relates to transdermal drug delivery, specifically to the use of novel combinations of excipients to improve drug delivery and compositions comprising the same.

### BACKGROUND

Transdermal drug delivery patches are drug reservoir materials that provide a discrete method of administering drugs to users. The patches adhere to the user's skin and drugs are released from the material and permeate the skin. It is important in such applications that the release of drugs from the patches and the transmittance of the drug through the skin is controlled to suite the required function. Some applications require drugs to be released slowly for a long duration whilst others require a swift delivery of drugs to the user.

In order to effect this, the materials from which the patches are made are carefully chosen to achieve the best results. However, varying the material composition alone is often insufficient to achieve the desired drug delivery profile. Accordingly, it is often the case that additional excipients are introduced into transdermal drug delivery patches to improve, or at least fine tune, the drug delivery profiles of patches.

The underlying reasons why certain excipients promote certain changes in the drug delivery profile is not entirely well understood. Excipients can be provided as a vehicle to improve transmission across the lipophilic skin barrier for hydrophilic drugs. Others can be used to change the polarity and pH of drugs for delivery.

It is an object of the invention to provide improved excipients for use in transdermal drug delivery.

### SUMMARY OF INVENTION

There is provided in a first aspect of the invention, a first composition for transdermal drug delivery comprising: a drug for transdermal drug delivery; and a first excipient, the excipient comprising an alkoxy alcohol and a glycol.

The inventors have found that above first excipient of alkoxy alcohols and glycols provides excellent improvements in the drug delivery properties of transdermal patches. Moreover, the improvements seen with these combinations are surprising as they are greater than the mere additive effect of the excipient components alone. Indeed, the reason why this synergistic effect is seen is not well understood.

There is no particular restriction on the type of drug with which the first excipients may be paired. However, it is typically the case that the drugs used are hydrophobic. Typical examples of hydrophobic drugs include Artemisinin, artesunate, aspirin, azathioprine, bisoprol, buprenorphine, calitrol, calciferol, capsaicin, carbamazepine, chlorhexidine, clobetasone butyrate, clonidine, clotrimazole, cyclosporine, dexamethasone, dicflucortolone valerate, diclofenac epolamine, ergotamine, β-estradiol, fenbufen, fentanyl, flurbiprofen, gestodene, hydrocortisone, ibuprofen, indomethacin, iodine, ivermectin, ketoprofen, lamotrigine, levomenthol, levonorgestrel, loratidine, melatonin, naproxen, norelgestromin, norethisterone, penicillin, piroxicam, pramipexole, praziquantel, prednisolone prilocaine, progesterone, propylthiouracil, quinidine, risperidone, salbutamol, methyl salicylate, salsalate, saquinavir, simvastatin, teriparatide, testosterone, triamcinolone and trimethoprim. It may be the case that the drug is ibuprofen.

Alternatively, the drug may be hydrophilic. Typical examples of hydrophobic drugs include Acyclovir, allopurinol, amoxicillin, caffeine, ceftriaxone, cisplatin, cyclophosphamide, dopamine, dopamine hydrochloride, doxycycline, fluloxetine, fluorourcil, gabapentin, gentamycin, glucose, lamivudine, lidocaine, methotrexate, nicotine, nystatin, paracetamol, penicillamine, silver nitrate, sufentanil citrate, temozolomide, tetracycline, triamcinolone, vitamin B12. It may be the case that the drug is lidocaine.

The drug is typically present in the first composition in an amount in the range of 3 to 40% by weight of the first composition, more typically 5 to 30% by weight of the first composition, more typically still 8 to 20% by weight of the first composition, even more typically 10 to 15% and often representing about 12.5% by weight of the first composition.

It has surprisingly been found that the combination of alkoxy alcohols and glycols is effective at improving the drug delivery of both hydrophobic and hydrophilic drugs.

It is often the case that the alkoxy alcohol is in the range C₂-C₁₀, more typically C₃-C₉, more typically C₄-C₈. The alkoxy alcohol may be in the range C₅-C₇ and is often a C₆ alkoxy alcohol. Typically the alkoxy alcohol is an ethoxy alcohol. The term, “ethoxy alcohol” as used herein is intended to mean, “an alcohol comprising an ethoxy group within the chain”. A typical example of an alkoxy alcohol suitable for use with the invention is transcutol, otherwise known as 2-(2-ethoxyethoxy)ethanol.

The glycol used in the present invention is typically a C₂-C₆ glycol. Usually, the glycol will be in the range C₃-C₄ and most typically the glycol is propylene glycol.

There is no particular restriction on the relative ratios of the two components. However, it is typically the case that the alkoxy alcohol is present in the range 1% to 40% by weight of the first composition, more typically 1 to 30% by weight of the first composition. More usually, the alkoxy alcohol is present in the range 1% to 20% by weight of the first composition and even more usually the alkoxy alcohol is present in the range 1% to 10% by weight of the first composition an amount equal to about 5% by weight of the first composition.

It is typically the case that the glycol is present in the range 1% to 40% by weight of the first composition, more typically 1 to 30% by weight of the first composition. More usually, the glycol is present in the range 1% to 20% by weight of the first composition, more typically the glycol is present in the range 1% to 10% by weight of the first composition and even more usually the glycol is present in an amount equal to about 5% by weight of the first composition.

The ratio of alkoxy alcohol to glycol is often in the range 1:10 to 10:1, more typically in the range of 1:5 to 5:1 and more typically still in the range 1:2 to 2:1. Further, it is often the case that the alkoxy alcohol and glycol are present in a ratio of about 1:1.

The first composition may further comprise a preservative to improve shelf-life of the composition and more importantly the patches into which the composition is incorporated. A typical example of such materials include aryl alcohols, such benzyl alcohol.

Other components may also be introduced into the first composition. For instance fatty acid, fatty alcohol and/or fatty ester may be introduced. Typically, fatty alcohol or fatty ester will be used as fatty acids can cause problems if they are included within the first composition during the curing process. Whilst there is no particular limitation on the choice of fatty alcohol used in the invention, a typical example of fatty alcohols is octadecanol. Similarly, the fatty esters are not particularly restricted but a typical example is isopropyl myristate.

Often, the first composition will comprise the fatty acid, fatty alcohol or fatty ester in the range 1 to 10% by weight of the first composition, more typically 1 to 5% by weight of the first composition and more typically still in the range 1 to 2% by weight of the first composition.

Polymers may also be incorporated into the excipient such as polyethylene glycol. These can also advantageously augment the properties of the excipients described herein, especially when provided in combination with the above mentioned fatty acids, fatty alcohols or fatty esters.

Additional additives may be introduced into the composition as would be familiar to a person skilled in the such as pH modifiers, surfactants and adhesives provided that said additional components do not interfere with the drug delivery properties of the composition.

There is provided in a second aspect of the invention, the use of the first excipient as permeability enhancer for transdermal drug delivery. The inventors have found that the first excipient is surprisingly effective at promote transdermal drug delivery.

Typically the use of the second aspect of the invention is in conjunction with a hydrophobic drug such as those described above with respect to the first aspect of the invention, in particular ibuprofen. Alternatively, the use of the second aspect of the invention may be in conjunction with a hydrophilic drug such as those described above with respect to the first aspect of the invention, in particular lidocaine.

There is also provided in a third aspect of the invention, a second composition for transdermal drug delivery comprising: a drug for transdermal drug delivery and a second excipient, wherein the second excipient comprises an alkoxy alcohol and a sulfoxide. The inventors have also found that the combination of alkoxy alcohol and sulfoxides also provides a surprising improvement in drug delivery properties.

The alkoxy alcohol used in the second excipient are as defined with respect to the first aspect of the invention. The sulfoxide used in the second excipient is typically an alkyl sulfoxide, usually a low alkyl sulfoxide such as dimethylsulfoxide (DMSO).

Typically, the sulfoxide is present in the range 1 to 40% by weight of the second composition, more typically 1 to 30% by weight of the second composition, more typically still 1 to 20% by weight of the second composition and even more typically 1 to 10% by weight of the second composition. Often the sulfoxide will be provided in an amount of about 5% by weight of the second composition. Further, the ratio of alkoxy alcohol to sulfoxide is in the range 1:10 to 10:1, more often in the range 1:5 to 5:1 and more typically still in the range 1:2 to 2:1. It is most often the case that the ratio of alkoxy alcohol to sulfoxide is about 1:1.

The drug used in the composition of the third aspect of the invention is typically hydrophilic. Typical examples of hydrophobic drugs include acyclovir, allopurinol, amoxicillin, caffeine, ceftriaxone, cisplatin, cyclophosphamide, dopamine, dopamine hydrochloride, doxycycline, fluloxetine, fluorourcil, gabapentin, gentamycin, glucose, lamivudine, lidocaine, methotrexate, nicotine, nystatin, paracetamol, penicillamine, silver nitrate, sufentanil citrate, temozolomide, tetracycline, triamcinolone and vitamin B12. It may be the case that the drug is lidocaine.

The drug is typically present in the second composition in an amount in the range of 3 to 40% by weight of the second composition, more typically 5 to 30% by weight of the second composition, more typically still 8 to 20% by weight of the second composition, even more typically 10 to 150% and often representing about 12.5% by weight of the second composition.

The second excipient may further comprise a preservative and/or comprise one or more fatty acid, fatty alcohol or fatty ester as defined in the first aspect of the invention.

There is also provided in a fourth aspect of the invention, the use of the second excipient as permeability enhancer for transdermal drug delivery. The inventors have found that the described excipient is surprisingly effective at promote transdermal drug delivery.

Typically, the use of the fourth aspect of the invention is in conjunction with a hydrophilic drug such as those described above with respect to the third aspect of the invention, in particular lidocaine.

Also provided herein in a fifth aspect of the invention a transdermal drug delivery patch comprising the composition according to the first aspect of the invention of the third aspect of the invention. As explained above, the claimed excipients promote beneficial drug release properties from transdermal delivery patches.

The first and second composition are typically each independently present in the patch in an amount in the range of 1 to 20% by total weight of the patch, more typically in the range of 2 to 15% by total weight of the patch, more typically still in the range of 3 to 10% by total weight of the patch and often in the range of 4 to 8% by total weight of the patch. Most often the patch comprises about 5% of first and second composition by the total weight of the patch.

There is no particular restriction on the kind of patches with which the excipients may be paired. However, it is typically the case that the patches comprise a crossed-linked silyl-containing polymer.

It is often the case that the crossed-linked silyl containing polymer is selected from: silyl-containing polyethers, silyl-containing polyurethanes, silyl-containing polyesters, silyl-containing polycarbonates, co-polymers thereof and/or combinations thereof.

The co-polymers may be block copolymers, random copolymers, alternating copolymers, graft copolymers or combinations thereof. More often than not the copolymers will be block copolymers.

The silyl-containing polymers are typically cross-linked to form a matrix. This of the above mentioned polymers has been found by the inventors to be particularly effective at storing compounds for drug delivery to the skin and also releases compounds gradually over a prolonged period of time, especially when combined with the claimed excipients and compositions of the invention. Further, the adhesive properties of the composition are not compromised by the addition of drugs, the claimed excipients or other common additives.

Examples of typical silyl-containing polymers suitable for use with the invention are disclosed in EP 2 235 133, EP 2 468 783, EP 2 865 728, EP 2 889 349, WO 2013 136108 and EP 2 889 348. In particular, those silyl-containing polymers described in EP 2 889 349 and EP 2 889 348.

It is typically the case that the patch includes a compatible tackifying resin. This improves the adhesive properties of the composition and allows the composition to be formulated into a pressure sensitive adhesive (PSA).

The tackifying resin may be selected from: phenol modified terpene resins (typically polyterpenes), hydrocarbon resins (typically where the hydrocarbons have an aromatic character, i.e. comprise one or more aromatic groups), rosin ester resins, modified rosin ester resins and acrylic resins. Typically, the phenol modified terpene resins have a softening point from, 70° C. to 150° C., or more typically 110° C. to 130° C.; the hydrocarbon resins have a softening point in the range 10° C. to 150° C. and more typically 70° C. to 120° C.; and the rosin ester resins have a softening point in the range 10° C. to 130° C., more typically 90° C. to 110° C.

The softening point of the silyl-containing polymer and/or of the tackifying resin can be measured according to ASTM E28 standard.

The tackifying resins are typically compatible with the skin and do not cause irritation, and are substantially non-cytotoxic. Further, the tackifying resins are typically resistant to degradation. Where the tackifying resins do break down over time (e.g. due to photolysis or hydrolysis during use or storage) it is typically the case that the breakdown products are substantially non-toxic and typically do not penetrate the skin.

Typically, the phenol modified terpene resins are obtained by polymerization of terpene hydrocarbons and phenols, in the presence of Friedel-Crafts catalysts.

According to one embodiment, hydrocarbon resins are selected from: resins obtained by a process comprising the polymerization or co-polymerization of [alpha]-methyl-styrene, said process possibly also including a reaction with phenols, resins obtained by hydrogenation, polymerization or copolymerization (with an aromatic hydrocarbon) of mixtures of unsaturated aliphatic hydrocarbons having less than or equal to 10 carbon atoms derived from petroleum fractions, optionally grafted with maleic anhydride, terpene resins, generally resulting from the polymerization of terpene hydrocarbons such as, for example, monoterpene (or pinene) in the presence of Friedel-Crafts catalysts, copolymers based on natural terpenes, for example styrene/terpene, [alpha]-methylstyrene/terpene and vinyltoluene/terpene.

According to one embodiment, rosin ester resins are selected from natural or modified rosins, such as for example the rosin extracted from pine gum, wood rosin extracted from tree roots and their derivatives that are hydrogenated, dimerized, polymerized or esterified by monoalcohols or polyols such as glycerol.

According to one embodiment, the molecular weight of the non-acrylic resins as above-disclosed is less than or equal to 10,000 Da, typically less than or equal to 2,000 Da, more typically less than or equal to 1,000 Da.

An acrylic resin is defined as a polymer or oligomer built with a significant amount of (meth)acrylic and/or (meth)acrylate monomers, usually at least 5% weight/weight (w/w), more often at least 10% w/w, still more usually at least 20% w/w, typically at least 30% w/w in the polymeric chain.

According to one embodiment (meth)acrylic monomers are chosen from acrylic acid, methacrylic acid, methyl acrylate, methyl methacrylate, ethyl acrylate, ethyl methacrylate, butyl acrylate, butyl methacrylate, isobutyl acrylate, isobutyl methacrylate, n-hexyl acrylate, n-hexyl methacrylate, 2-ethylhexyl acrylate, ethylhexyl methacrylate, n-heptyl acrylate, n-heptyl methacrylate, stearyl acrylate, stearylmethacrylate, glycidyl methacrylate, alkyl crotonates, vinyl acetate, di-n-butyl maleate, di-octylmaleate, acetoacetoxyethyl methacrylate, acetoacetoxyethyl acrylate, acetoacetoxypropyl methacrylate, acetoacetoxypropyl acrylate, diacetone acrylamide, acrylamide, methacrylamide, hydroxyethyl methacrylate, hydroxyethyl acrylate, allyl methacrylate, tetrahydrofurfuryl methacrylate, tetrahydrofurfuryl acrylate, cyclohexylmethacrylate, cyclohexyl acrylate, n-hexyl acrylate, n-hexyl methacrylate, 2-ethoxyethyl acrylate, 2-ethoxyethyl methacrylate, isodecyl methacrylate, isodecyl acrylate, 2-methoxy acrylate, 2-methoxy methacrylate, 2-(2-ethoxyethoxy) ethylacrylate, 2-phenoxyethyl acrylate, 2-phenoxyethyl methacrylate, isobornyl acrylate, isobornyl methacrylate, caprolactone acrylate, caprolactone methacrylate, polypropyleneglycol monoacrylate, polypropyleneglycol monomethacrylate, polyethylene glycol (400) acrylate, polypropyleneglycol (400) methacrylate, benzyl acrylate, benzylmethacrylate, N-vinyl pyrrolidone or N-vinyl lactam.

Typically, (meth)acrylic monomers have up to 20 carbon atoms, and are typically selected from acrylic acid, methacrylic acid, butyl acrylate, 2-ethylhexyl acrylate and hydroxyethyl acrylate.

According to one embodiment, acrylic resins are selected from polymers containing at least one (meth)acrylic function or chain part and at least one hydrocarbon chain part, said polymers can be in the form of copolymers, grafted or reacted or block polymers.

The above described resins have a viscosity measured at 100° C. significantly greater or equal to 100 Pa·s, and less than or equal to 100 Pa·s at 150° C. The acrylate resins may comprise repeating units of at least one hydrocarbon monomer and at least one acrylate monomer. Hydrocarbon monomers are selected from the group consisting of styrene, alpha-methyl styrene, vinyl toluene, indene, methylindene, divinylbenzene, dicyclopentadiene, and methyl-dicyclopentadiene, and polymerizable monomers contained in C5-pyperylenic and C5-isoprene and C9-aromatic available streams from the petrochemical industry. Those hydrocarbon monomers are usually polymerized together in various ratios by cationic polymerization using Lewis acid catalysts. Acrylate monomers have the general formula Rₐ—CH═CR_{b}—COOR_{c} wherein Rₐ, R_{b}, R_{c} are independently selected from hydrogen, aliphatic groups, and aromatic groups. Acrylate monomers are selected from the group consisting of methyl acrylate, acrylic acid, methacrylic acid, methyl methacrylate, ethyl acrylate, ethyl methacrylate, butyl acrylate, butyl methacrylate, isobutyl acrylate, isobutyl methacrylate, n-hexyl acrylate, n-hexyl methacrylate, ethylhexyl acrylate, ethylhexyl methacrylate, n-heptyl acrylate, n-heptyl methacrylate, 2-methyl heptyl(meth)acrylate, octyl acrylate, octyl methacrylate, isooctyl(meth)acrylate, n-nonyl(meth)acrylate, iso-nonyl(meth)acrylate, decyl(meth)acrylate, isodecyl acrylate, isodecyl methacrylate, dodecyl(meth)acrylate, isobornyl(meth)acrylate, lauryl methacrylate, lauryl acrylate, tridecyl acrylate, tridecyl methacrylate, stearyl acrylate, stearylmethacrylate, glycidylmethacrylate, alkyl crotonates, vinyl acetate, di-n-butylmaleate, di-octylmaleate, acetoacetoxyethyl methacrylate, acetoacetoxyethyl acrylate, acetoacetoxypropyl methacrylate, acetoacetoxypropyl acrylate, diacetone acrylamide, acrylamide, methacrylamide, hydroxy ethylmethacrylate, hydroxyethyl acrylate, allyl methacrylate, tetrahydrofurfuryl methacrylate, tetrahydrofurfuryl acrylate, cyclohexyl methacrylate, cyclohexyl acrylate, 2-ethoxyethyl acrylate, 2-ethoxyethyl methacrylate, isodecyl methacrylate, isodecyl acrylate, 2-methoxy acrylate, 2-methoxy methacrylate, 2-(2-ethoxyethoxy)ethylacrylate, 2-phenoxyethyl acrylate, 2-phenoxyethyl methacrylate, caprolactone acrylate, caprolactone methacrylate, polypropyleneglycol monoacrylate, polypropyleneglycol monomethacrylate, poyethyleneglycol(400)acrylate, polypropyleneglycol(400) methacrylate, benzyl acrylate, benzylmethacrylate, sodium I-allyloxy-2-hydroylpropyl sulfonate, acrylonitrile, and mixtures thereof.

Typically hydrocarbon monomers are selected among the group of aromatic monomers or polymerizable monomers from the C9-aromatic stream from petrochemical sources; of dicyclopentadiene or polymerizable monomers from the C5-pyperylene or C5-isoprene stream from petrochemical sources.

Usually acrylate monomers are acrylic acid and 2-ethylhexyl acrylate, hydroxyethyl acrylate, methacrylic acid, butyl acrylate. Softening point of such resins are typically from room temperature up to 180° C., molecular weights range in weight average is typically from 200 to 25000 Daltons, and acid number typically ranging from 0 to 300 mg KOH g⁻¹. Typical resins would have molecular weight less than or equal to 10,000 Daltons, more usually less than or equal to 2,000 Da, most typically less than or equal to 1,000 Da; softening point less than or equal to 150° C., more typically less than or equal to 120° C., most typically ranging from 70 to 120° C.; acid number less than or equal to 150 mg KOH g⁻¹, more typically less than or equal to 100 mg KOH g⁻¹, most typically from 10 to 100 mg KOH g⁻¹.

According to one embodiment, the molecular weight of an acrylic resin is less than or equal to 300,000 when only one resin is present in the composition, usually less than or equal to 100,000, most typically less than or equal to 20,000.

A non-acrylic resin can still contain some acrylic functions in a non-significant quantity, either being part of the polymerization chemical reaction, or as grafted or functionalized groups onto monomers or onto the polymeric chains.

The ratio of tackifying resin to silyl-containing polymer is typically in the range 1:10 to 10:1, more typically, 1:2 to 2:1 and is typically about 1:1. The composition typically comprises: a) from 20 to 85% by weight, more typically 30 to 60% by weight of at least one silyl-containing polymer as described above; and b) from 15 to 80% by weight, or more typically 30 to 60% by weight of at least one tackifying resin. Typically the composition comprises about 50% silyl-containing polymer and about 50% tackifying resin.

As used herein, reference the delivery of drugs “to the skin” it is meant that the drugs are administered either: onto the surface of the skin; into the skin; or delivered to the body transdermally i.e. through the skin and into the blood stream.

The term “drug” as used herein is intended to refer to a biologically active substance. There is no particular limitation on the type of compound from which the drug is made. The drugs used with the present invention are typically molecules with low molecular weight, especially where the drug is intended for transdermal delivery. However larger molecules and macromolecules are also envisaged including biological compounds such as peptides and proteins. The term “drug” is also intended to encompass pharmaceutically acceptable salts of biologically active substances. It is also envisaged that the drug may provide a physical effect on the body, such as heating or cooling, which may have a therapeutic effect.

The term, “small molecule drugs” is intended to encompass those compounds typically produced by synthetic chemical processes having a molecular weight typically less than 1000 Da, more typically less than 700 Da.

The term “polymer” is intended to refer to macromolecules comprised of a plurality of repeating monomer units, typically having a weight average molecular weight of greater than 600 Da, preferably greater than 2000 Da.

The term “cross-linked” as used herein is intended to refer to the covalent interconnection of polymers within a composition either directly (polymer to polymer) or indirectly (polymer to intermediate bridging group to polymer) typically as a result of a reaction between particular polymer side groups and other corresponding side groups on adjacent polymers or intermediate bridging groups. This may be achieved using a catalyst and/or with the presence of co-reactants, such as water. Further, elevated temperatures, radiation such as ultraviolet (UV) radiation or electron-beam (EB) radiation may be used to promote the cross-linking reaction. Where a catalyst is used, at least one catalyst is typically present in the composition in an amount in the range 0.001 to 5% by weight, more typically 0.01 to 3% by weight of the composition. The catalyst may remain in the composition or may be used up in the cross-linking process.

The term “curing” as used herein is to be understood as “cross-linking” (as described above) the components of a composition together until the desired properties of the cured material are achieved. This cross-linking in the present invention typically occurs between silyl groups of the silyl-containing polymers described above.

It is typically the case that the silyl-containing polymers describe above will have a weight average molecular weight in the range 700 Da to 250 kDa, more typically from 6 kDa to 100 kDa and even more typically from 10 kDa to 50 kDa.

The dispersity of the silyl-containing polymers is typically less than 3, more typically less than 2 and is most typically in the range 1.0 to 1.6, typically 1.1 to 1.4.

Any numerical value provided herein is intended to be modified by the term “about”. Further, the disclosure of a range is intended to disclose the range, the specific values therebetween the limits of the range and especially the integers between said limits.

In addition, although features may be described as “comprising” part of the invention, all the features described herein may also be considered as “consisting of” or “consisting essentially of” part of the invention.

The invention will now be described with respect to specific examples.

### DESCRIPTION OF FIGURES

FIG. 1 shows ibuprofen release from pressure sensitive adhesive.

FIG. 2 shows permeation of ibuprofen through Strat-M membranes.

FIG. 3 shows permeation of ibuprofen through Strat-M membranes with propylene glycol in formulations.

FIG. 4 shows permeation of ibuprofen through Strat-M membranes with Transcutol, i.e. 2-(2-ethoxyethoxy) ethanol, in formulations.

FIG. 5 shows permeation of ibuprofen through Strat-M membranes enhanced with the mixture of excipients.

FIG. 6 shows ibuprofen permeation with fatty alcohols.

FIG. 7 shows permeation of ibuprofen through Strat-M membranes using optimised mixtures of excipients.

FIG. 8 shows Cumulative amount of lidocaine utilising hydrophilic excipients 10 wt %.

FIG. 9 shows Flux values of lidocaine utilising hydrophilic excipients 10 wt %.

FIG. 10 shows Permeation of lidocaine utilising hydrophilic excipients 10 wt % across Strat-M membrane.

FIG. 11 shows Cumulative amount of lidocaine utilising excipient mixtures 5 wt % for each compound.

FIG. 12 shows Flux values of lidocaine utilising excipient mixtures 5 wt % for each compound.

FIG. 13 shows Permeation of lidocaine utilising mixtures of hydrophilic excipients (5 wt %) across Strat-M membrane.

FIG. 14 shows Adhesion study of the lidocaine TEPI patch utilising mixtures of hydrophilic excipients (5 wt %) across Strat-M membrane.

FIG. 15 shows Cumulative amount of lidocaine utilising mixtures of glycols with fatty compounds.

FIG. 16 shows Flux values of lidocaine utilising mixtures of glycols with fatty compounds.

FIG. 17 shows Permeation of lidocaine utilising mixtures of glycols with fatty compounds across Strat-M membrane.

FIG. 18 shows Permeation of lidocaine utilising mixtures of hydrophilic excipients (5 wt %) across Strat-M membrane.

FIG. 19 shows Permeation of lidocaine utilising mixtures of hydrophilic excipients (5 wt %) across Strat-M membrane.

FIG. 20 shows Permeation of lidocaine utilising mixtures of hydrophilic excipients (5 wt %) across Strat-M membrane.

### EXAMPLES

Principles of Observation

Prior to determination of active pharmaceutical ingredient (API) diffusion across skin mimicking membranes, a passive release of ibuprofen from the pressure sensitive adhesive must be verified. This study allows for confirmation that the cured adhesive is a reservoir for API and does not limit its use as a transdermal drug delivery system. Such an experiment can be performed using non-rate-limiting membranes, for example porous silicone, Nylon or polytetrafluoroethylene (PTFE). In this study, Nylon was employed as the test model for passive release.

Pressure Sensitive Adhesive

The pressure sensitive adhesive (PSA) used in the below experiments were cross-linked silyl containing polyurethanes synthesised from polyethers/polyesters and diisocyanates.

Said polymers were provided together with a tackifying resin. Examples of the types of materials used as the pressure sensitive adhesive are described in WO 2013/136108. Such adhesives are known in the art.

Results

All further mentioned patches contain 10 wt % of API. Unless stated otherwise, 6 sample patches were analysed in parallel. In this particular case, patches did not contain any excipients, apart from benzyl alcohol as the preservative. Experimental conditions are listed below.

Patch components: Pressure sensitive adhesive, ibuprofen, benzyl alcohol.
Patch thickness: 300 μm
Membrane type: Nylon, pore size 0.45 μm
Apparatus: diffusion cells
Acceptor medium: mixture of pH 7.4 PBS/2-(2-ethoxyethoxy) ethanol (90/10 vol.)

### Temperature: 32±1° C.

Duration: 12 hours
Sample taken at: 1, 2, 4, 6, 8, 10 and 12 hours
Analysis: validated HPLC method

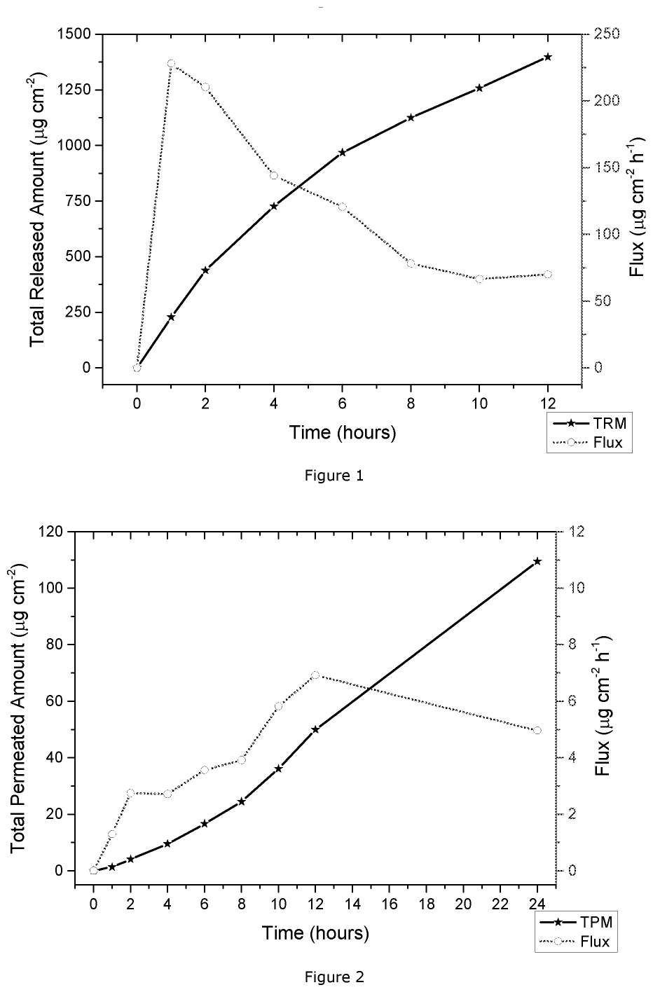

Obtained data is outlined in Table 1. Values of total released amount and flux across the membrane was plotted against sampling time and displayed in FIG. 1.

TABLE 1
Passive release of ibuprofen from pressure sensitive adhesive.

| | Time, | Total Released Amount, | Flux, |
|---|---|---|---|
| Sample No | hours | μg cm⁻² | μg cm⁻² h⁻¹ |
| 1 | 1 | 227.93 | 227.93 |
| 2 | 2 | 438.23 | 210.30 |
| 3 | 4 | 726.09 | 143.93 |
| 4 | 6 | 967.49 | 120.70 |
| 5 | 8 | 1124.13 | 78.32 |
| 6 | 10 | 1257.46 | 66.66 |
| 7 | 12 | 1397.34 | 69.94 |

The data shows clearly that there are no barriers for the API being released from the patch. Both parameters are shifted as expected: total amount is growing, while flux reduces due to the decrease of ibuprofen in the patch.

Judged by this criterion, ibuprofen can easily be released from patches which make them an excellent reservoir for a transdermal drug delivery system. However, chemical and physical properties of ibuprofen should be taken into account in context of its permeation through a real skin or mimicking synthetic analogues due to the complexity of the phenomenon.

Experimental Approach

In order to determine the necessity for further modification of ibuprofen patches with excipients, permeation rates of the API were observed using Strat-M membranes. Strat-M membranes are produced by Merck Millipore as a synthetic transdermal diffusion model to avoid animal or human skin testing. There is a correlation tool on a dedicated website allowing a comparison with human skin. Permeation profiles can vary depending on a drug, but it proves that Strat-M membranes can be employed as a reliable evaluation of any transdermal system in terms of human skin penetration. It must be said that penetration profiles in the case of human skin may differ from the aforementioned membrane, but allows predicting the efficacy of patches with certain reliability.

Patches tested in this study are from the same batch as employed in release experiments and were run under the same conditions. Obtained data is summarised in Table 2. Permeation profiles and release profiles of ibuprofen through Strat-M membranes are depicted in FIGS. 1 and 2, respectively.

TABLE 2
Comparison of release and permeation profiles
of ibuprofen from TEPI patches

| Sample | Time, h | Total Amount, μg cm⁻² | | Flux, μg cm⁻² h⁻¹ | |
|---|---|---|---|---|---|
| Membrane | | Nylon | Strat-M | Nylon | Strat-M |
| 1 | 1 | 227.93 | 1.291 | 227.93 | 1.29 |
| 2 | 2 | 438.23 | 4.044 | 210.30 | 2.75 |
| 3 | 4 | 726.09 | 9.466 | 143.93 | 2.71 |
| 4 | 6 | 967.49 | 16.595 | 120.70 | 3.56 |
| 5 | 8 | 1124.13 | 24.424 | 78.32 | 3.91 |
| 6 | 10 | 1257.46 | 36.066 | 66.66 | 5.82 |
| 7 | 12 | 1397.34 | 49.915 | 69.94 | 6.92 |
| 8 | 24 | 1650.21 | 109.467 | 21.07 | 4.96 |

As it can be deduced from the above presented data, release and permeation profiles are remarkably different. This can be explained by a penetration barrier for ibuprofen in the skin mimicking Strat-M membranes. Most probable cause of that phenomenon is the hydrophobicity of ibuprofen. Being virtually insoluble in water (21 μg mL⁻¹), ibuprofen cannot effectively penetrate across skin mimicking Strat-M membranes.

Various glycols, propylene glycol and diethylene glycol monoethyl ether (2-(2-ethoxyethoxy) ethanol) were tested to explore their effect on release. Mixtures of glycols and fatty acids, for example oleic acid, were also tested. It was found that even small amounts (1-3 wt %) of such compounds can multiply fluxes by factor of 10.

However, the use of any acidic compounds as excipients in patches was not considered as they can have an inhibiting role during the curing process. Therefore, for practical purposes, it was decided to replace fatty acids with fatty alcohols in formulations. Experiments were conducted using octadecanol and isopropyl myristate and results described below.

### Example 1: Propylene Glycol vs. 2-(2-ethoxyethoxy) ethanol [Strat-M]

Patches employed in this study contained 2.5, 5.0, 7.5 and 10 wt % of propylene glycol or 2-(2-ethoxyethoxy) ethanol. Strat-M membranes were chosen as the test model. The mass fraction of benzyl alcohol (2 wt %) was kept constant in all formulations. Obtained results were compared to the pure patch mentioned in Section 3.2. Experimental conditions are listed below.

Patch components: Pressure sensitive adhesive, ibuprofen, benzyl alcohol, propylene glycol, 2-(2-ethoxyethoxy) ethanol
Patch thickness: 300 μm
Membrane type: Strat-M
Apparatus: diffusion cells
Acceptor medium: mixture of pH 7.4 PBS/2-(2-ethoxyethoxy) ethanol (90/10 vol.)

### Temperature: 32±1° C.

Duration: 24 hours
Sample taken at: 1, 2, 4, 6, 8, 10, 12 and 24 hours
Analysis: validated HPLC method

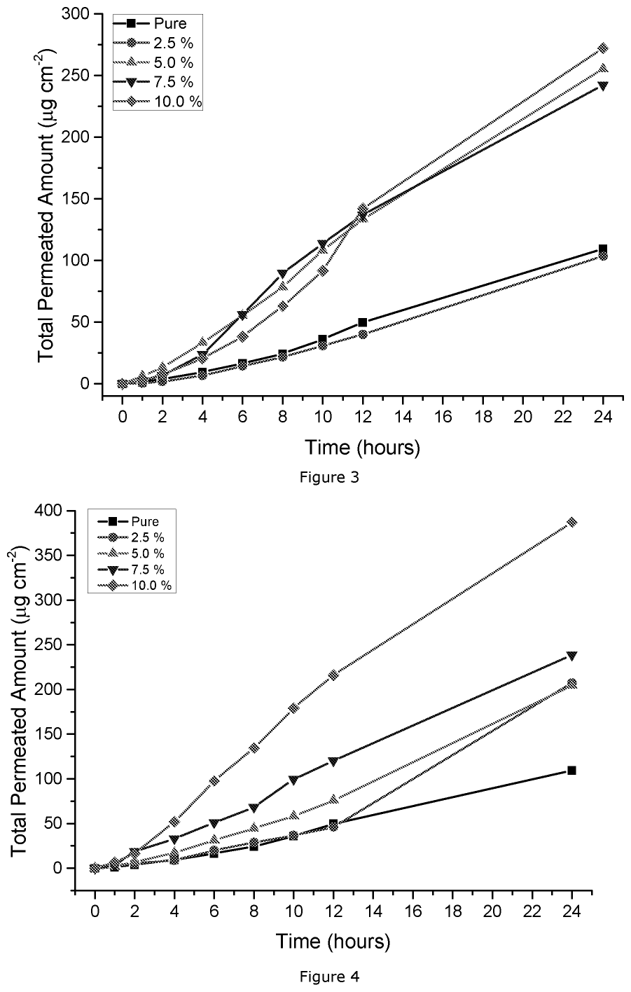

Obtained values for propylene glycol are summarised in Table 3 and graphically represented in FIG. 3. An effect of 2-(2-ethoxyethoxy) ethanol on API permeation is given in Table 4. A graph of total permeated amounts over the analysis period of time is depicted in FIG. 4.

TABLE 3
Ibuprofen permeation enhancement with propylene glycol.

| | | Total Permeated Amount, | | | | | | | | | |
|---|---|---|---|---|---|---|---|---|---|---|---|
| Sample | Time, h | μg cm⁻² | | | | | Flux, μg cm⁻² h⁻¹ | | | | |
| Excipient, wt % | | 0.0 | 2.5 | 5.0 | 7.5 | 10 | 0.0 | 2.5 | 5.0 | 7.5 | 10 |
| 1 | 1 | 1.29 | 0.50 | 6.11 | 2.51 | 2.57 | 1.29 | 0.50 | 6.11 | 2.51 | 2.57 |
| 2 | 2 | 4.04 | 1.76 | 13.42 | 6.82 | 7.56 | 2.75 | 1.26 | 7.30 | 4.31 | 4.99 |
| 3 | 4 | 9.47 | 6.66 | 33.34 | 23.77 | 20.53 | 2.71 | 2.45 | 9.96 | 8.47 | 6.49 |
| 4 | 6 | 16.60 | 14.39 | 55.39 | 56.34 | 38.26 | 3.56 | 3.86 | 11.03 | 16.29 | 8.86 |
| 5 | 8 | 24.42 | 21.75 | 78.35 | 89.92 | 62.92 | 3.91 | 3.68 | 11.48 | 16.79 | 12.33 |
| 6 | 10 | 36.07 | 30.89 | 108.27 | 113.85 | 91.64 | 5.82 | 4.57 | 14.96 | 11.97 | 14.36 |
| 7 | 12 | 49.91 | 40.05 | 133.43 | 137.04 | 142.16 | 6.92 | 4.58 | 12.58 | 11.59 | 25.26 |
| 8 | 24 | 109.47 | 103.69 | 255.43 | 242.40 | 272.25 | 4.96 | 5.30 | 10.17 | 8.78 | 10.84 |

TABLE 4
Ibuprofen permeation enhancement with 2-(2-ethoxyethoxy) ethanol.

| | | Total Permeated Amount, | | | | | | | | | |
|---|---|---|---|---|---|---|---|---|---|---|---|
| Sample | Time, h | μg cm⁻² | | | | | Flux, μg cm⁻² h⁻¹ | | | | |
| Excipient, wt % | | 0.0 | 2.5 | 5.0 | 7.5 | 10 | 0.0 | 2.5 | 5.0 | 7.5 | 10 |
| 1 | 1 | 1.29 | 3.32 | 1.90 | 3.69 | 6.72 | 1.29 | 3.32 | 1.90 | 3.69 | 6.72 |
| 2 | 2 | 4.04 | 5.93 | 6.94 | 18.95 | 16.59 | 2.75 | 2.61 | 5.04 | 15.26 | 9.87 |
| 3 | 4 | 9.47 | 9.13 | 17.58 | 33.12 | 52.19 | 2.71 | 1.60 | 5.32 | 7.08 | 17.80 |
| 4 | 6 | 16.60 | 20.08 | 31.52 | 51.17 | 97.72 | 3.56 | 5.48 | 6.97 | 9.03 | 22.77 |
| 5 | 8 | 24.42 | 28.81 | 44.60 | 68.46 | 134.45 | 3.91 | 4.36 | 6.54 | 8.65 | 18.37 |
| 6 | 10 | 36.07 | 36.66 | 58.17 | 99.65 | 179.25 | 5.82 | 3.93 | 6.79 | 15.59 | 22.40 |
| 7 | 12 | 49.91 | 46.10 | 75.97 | 120.44 | 216.13 | 6.92 | 4.72 | 8.90 | 10.40 | 18.44 |
| 8 | 24 | 109.47 | 207.38 | 204.53 | 238.86 | 387.26 | 4.96 | 13.44 | 10.71 | 9.87 | 14.26 |

From the data presented, it can be concluded that propylene glycol and 2-(2-ethoxyethoxy) ethanol enhance permeation differently. For instance, 2.5 wt % of propylene glycol has no effect, while 5.0, 7.5 and 10.0 wt % do increase permeated amounts of API but the difference is marginal. 2.5 wt % of 2-(2-ethoxyethoxy) ethanol showed a definite increase of total permeated amount over 24 h being very similar to the pure formulation in 12 h. The increase of 2-(2-ethoxyethoxy) ethanol to 5.0 and 7.5 wt % enhanced the penetration of ibuprofen straight away resulting in a higher amount in 12 h, but did not significantly improved the performance over 24 h. In the case of 10 wt %, 2-(2-ethoxyethoxy) ethanol showed the best results.

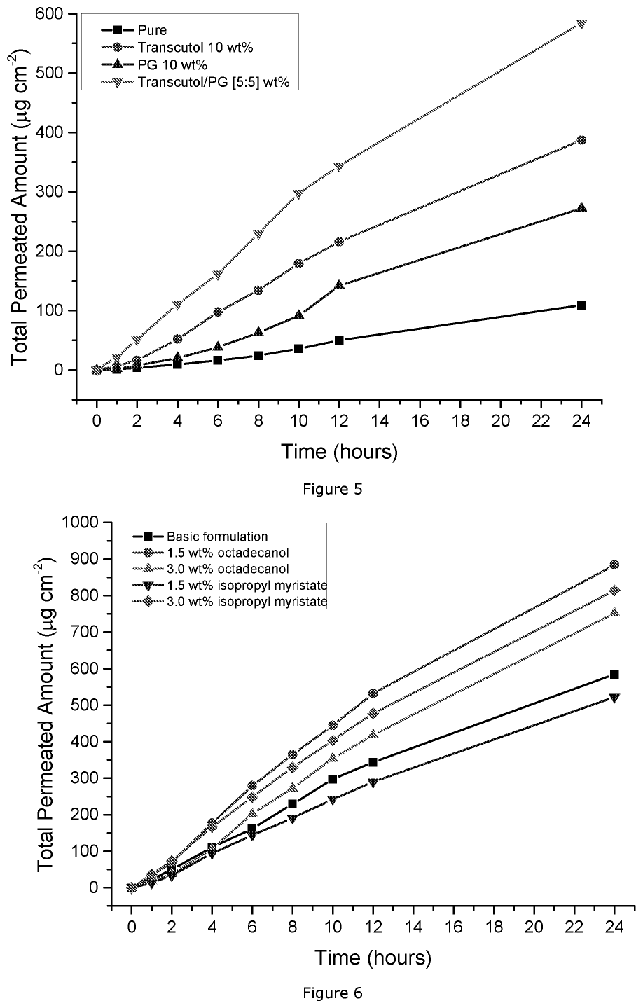

Considering our previous findings, we decided to keep the ratio of API to excipients 1:1 by weight and employ a mixture of propylene glycol and 2-(2-ethoxyethoxy) ethanol. Above presented data allows us to expect a mixture of 5 wt % of propylene glycol and 5 wt % of 2-(2-ethoxyethoxy) ethanol to be the most efficient. 2.5 wt % of propylene glycol is not efficient whilst higher amounts do not improve permeation significantly. 2-(2-ethoxyethoxy) ethanol seems to be potent with all tested mass fractions. Obtained data is outlined in Table 5 and graphically represented in FIG. 5.

TABLE 5
Permeation enhancement with the mixture of excipients.

| Sample | Time, h | Total Permeated Amount, μg cm⁻² | | | | Flux, μg cm⁻² h⁻¹ | | | |
|---|---|---|---|---|---|---|---|---|---|
| Excipient, wt % | | 0 | PG 10 | Tran. 10 | Mix. 5/5 | 0 | PG 5 | Tran. 10 | Mix. 5/5 |
| 1 | 1 | 1.29 | 2.57 | 6.72 | 21.94 | 1.29 | 2.57 | 6.72 | 21.94 |
| 2 | 2 | 4.04 | 7.56 | 16.59 | 51.24 | 2.75 | 4.99 | 9.87 | 29.30 |
| 3 | 4 | 9.47 | 20.53 | 52.19 | 111.08 | 2.71 | 6.49 | 17.80 | 29.92 |
| 4 | 6 | 16.60 | 38.26 | 97.72 | 161.77 | 3.56 | 8.86 | 22.77 | 25.34 |
| 5 | 8 | 24.42 | 62.92 | 134.45 | 229.74 | 3.91 | 12.33 | 18.37 | 33.98 |
| 6 | 10 | 36.07 | 91.64 | 179.25 | 297.93 | 5.82 | 14.36 | 22.40 | 34.10 |
| 7 | 12 | 49.91 | 142.16 | 216.13 | 343.93 | 6.92 | 25.26 | 18.44 | 23.00 |
| 8 | 24 | 109.47 | 272.25 | 387.26 | 584.61 | 4.96 | 10.84 | 14.26 | 20.06 |

As it can be seen in FIG. 5, a synergetic action of propylene glycol and 2-(2-ethoxyethoxy) ethanol remarkably accelerates the penetration of ibuprofen.

### Example 2: Octadecanol (OCD) Vs. Isopropyl Myristate (IPM) [Strat-M]

Patches analysed in this study were formulated using a basic formulation (5 wt % of propylene glycol, 5 wt % of 2-(2-ethoxyethoxy) ethanol, 2 wt % of benzyl alcohol) and 1.5-3 wt % of fatty alcohols. Strat-M membranes were chosen as the test model. Experimental conditions were kept the same as described above. Obtained results are summarised in Table 6 and depicted in FIG. 6.

TABLE 6
An effect of fatty alcohols on ibuprofen permeation.

| Sample | Time, h | Total Permeated Amount, μg cm⁻² | | | | Flux, μg cm⁻² h⁻¹ | | | |
|---|---|---|---|---|---|---|---|---|---|
| Excipient, wt % | | OD 1.5 | OD 3.0 | IPM 1.5 | IPM 3.0 | OD 1.5 | OD 3.0 | IPM 1.5 | IPM 3.0 |
| 1 | 1 | 34.57 | 18.56 | 13.77 | 36.88 | 34.57 | 18.56 | 13.77 | 36.88 |
| 2 | 2 | 69.70 | 38.64 | 34.31 | 73.92 | 35.13 | 20.08 | 20.54 | 37.03 |
| 3 | 4 | 177.00 | 106.88 | 94.46 | 166.43 | 53.65 | 34.12 | 30.08 | 46.26 |
| 4 | 6 | 279.60 | 202.06 | 144.66 | 248.55 | 51.30 | 47.59 | 25.10 | 41.06 |
| 5 | 8 | 365.56 | 272.90 | 191.10 | 329.61 | 42.98 | 35.42 | 23.22 | 40.53 |
| 6 | 10 | 444.96 | 354.41 | 242.58 | 403.94 | 39.70 | 40.75 | 25.74 | 37.16 |
| 7 | 12 | 532.12 | 418.44 | 290.04 | 477.44 | 43.58 | 32.02 | 23.73 | 36.75 |
| 8 | 24 | 883.87 | 752.30 | 522.06 | 814.51 | 29.31 | 27.82 | 19.34 | 28.09 |

The data suggests that fatty alcohols definitely improve the permeation of ibuprofen if compared to the basic formulation. Interestingly, patches containing 1.5 wt % of octadecanol are more efficient that 3.0 wt %, whilst isopropyl myristate exhibits an opposite behaviour.

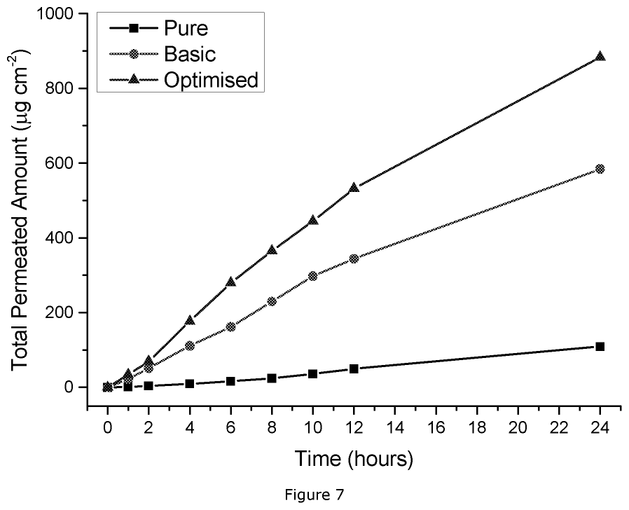

A summary of excipient optimisation is given in Table 7 and graphically represented in FIG. 7.

TABLE 7
Evolution of ibuprofen permeation with employed enhancers. Pure - no excipients
were used; Basic - mixture of propylene glycol and 2-(2-ethoxyethoxy) ethanol
5/5 wt %; Optimised - mixture of Basic and 1.5 wt % of octadecanol.

| Sample | Time, h | Total Permeated Amount, μg cm⁻² | | | Flux, μg cm⁻² h⁻¹ | | |
|---|---|---|---|---|---|---|---|
| Excipient, wt % | | 0 (Pure) | 10 (Basic) | 11.5 (Optimised) | 0 (Pure) | 10 (Basic) | 11.5 (Optimised) |
| 1 | 1 | 1.29 | 21.94 | 34.57 | 1.29 | 21.94 | 34.57 |
| 2 | 2 | 4.04 | 51.24 | 69.70 | 2.75 | 29.30 | 35.13 |
| 3 | 4 | 9.47 | 111.08 | 177.00 | 2.71 | 29.92 | 53.65 |
| 4 | 6 | 16.60 | 161.77 | 279.60 | 3.56 | 25.34 | 51.30 |
| 5 | 8 | 24.42 | 229.74 | 365.56 | 3.91 | 33.98 | 42.98 |
| 6 | 10 | 36.07 | 297.93 | 444.96 | 5.82 | 34.10 | 39.70 |
| 7 | 12 | 49.91 | 343.93 | 532.12 | 6.92 | 23.00 | 43.58 |
| 8 | 24 | 109.47 | 584.61 | 883.87 | 4.96 | 20.06 | 29.31 |

### Example 3: DMSO, 2-(2-Ethoxyethoxy) Ethanol, PEG₄₀₀, PG & Mixtures

Hydrophilic enhancers were employed including sulfoxides (DMSO), glycols and mixtures thereof. Their incorporation in formulations can assist by overcoming penetration limits and by tuning properties of the hydrophilic layer of the Strat-M membrane (or human skin). This can occur via saturation of that layer by these compounds or/and solvent drag. Among various glycols, polyethylene glycol 400 (PEG₄₀₀), propylene glycol and diethylene glycol monoethyl ether (2-(2-ethoxyethoxy) ethanol) have proved to be the most efficient. DMSO is a powerful aprotic solvent and it is often used in many areas of pharmaceutical sciences as a “universal solvent”.

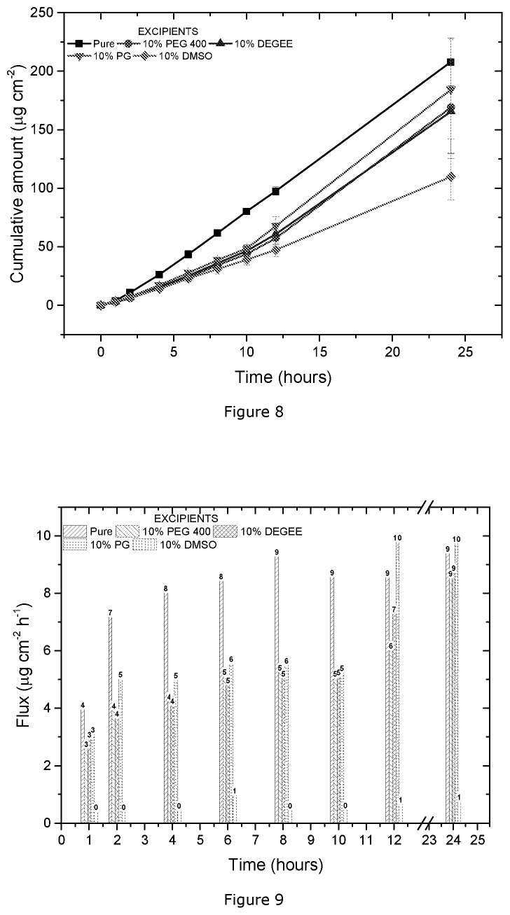

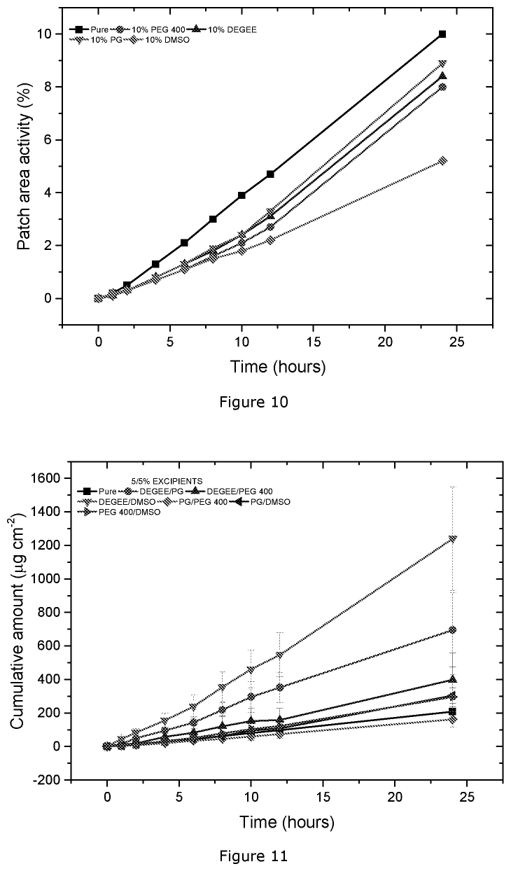

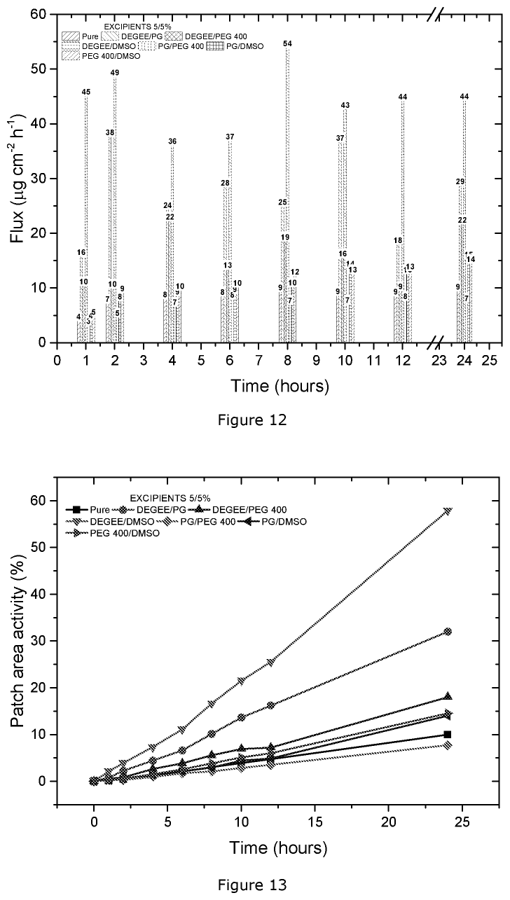

The effect of the aforementioned excipients on the lidocaine formulations was studied either separately (max 10 wt %) or as mixtures (max 5 wt % for each compound). An effect of DMSO and glycols addition to lidocaine formulations are given in FIGS. 8-13.

The data suggest that the addition of the excipients separately (10 wt %) does not enhance the delivery of lidocaine across the membrane and the permeation amounts are lower than the pure (no excipients) formulation (FIGS. 8-10). Interestingly, when a mixture of 5 wt % 2-(2-ethoxyethoxy) ethanol with 5 wt % of DMSO was employed the permeation increased approximately 6 times when compared to the pure, indicating that this particular mixture is the most efficient formulation (FIG. 11). The mixture of 2-(2-ethoxyethoxy) ethanol with propylene glycol shows also a significant increase. However, the rest formulations do not improve the permeation or they show only a marginal difference compared to the pure API formulation (FIGS. 11-13).

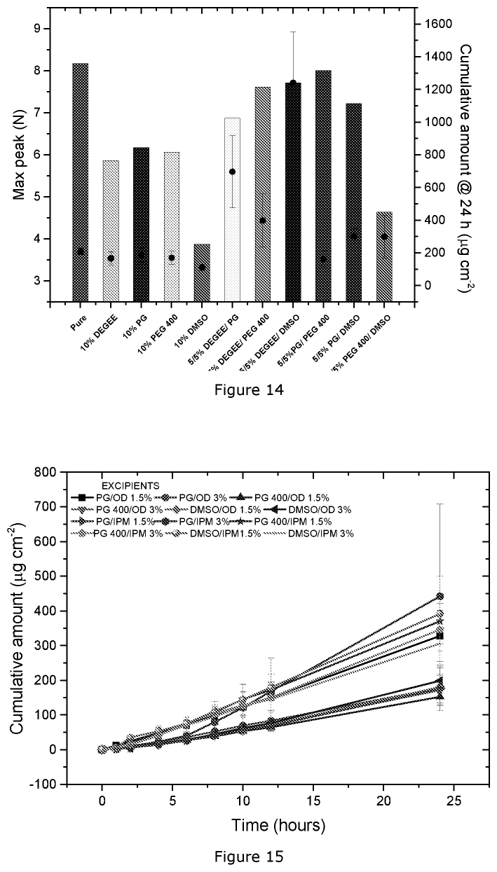

The impact of the addition of excipients on the adhesion was also determined employing loop tack test. FIG. 14 shows that there is only a marginal decrease on the adhesion of the most efficient formulations, 2-(2-ethoxyethoxy) ethanol/PG and 2-(2-ethoxyethoxy) ethanol/DMSO (yellow and dark blue traces respectively) when compared with the basic (red trace).

### Example 4: Octadecanol and Isopropyl Myristate (IPM)

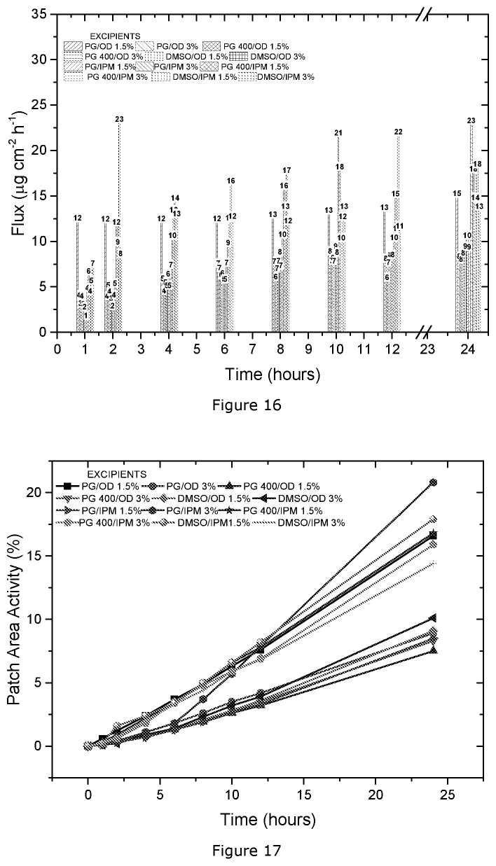

In addition to glycols, flux rates of some APIs (mainly lipophilic) can be increased using a mixture of glycols and fatty acids, for example oleic acid. It was reported that even small amounts (1-3 wt %) of such compounds can multiply fluxes by factor of 10. Octadecanol and isopropyl myristate were selected as test compounds (1.5 or 3 wt %) and the findings of this study are summarised in FIGS. 15-17.

The results for this part indicate that the addition of fatty compounds does not enhance the permeation of lidocaine across the Strat-M membrane significantly. Given the hydrophilic nature of the API these results were anticipated. IPM in any mixture increases the total permeation amount when compared to the pure formulation (cumulative amount 208 μg cm⁻²), however the difference is not significant. On the other hand, octadecanol gives lower values than the basic formulation for all combinations employed.

### Example 5: Optimised Formulations

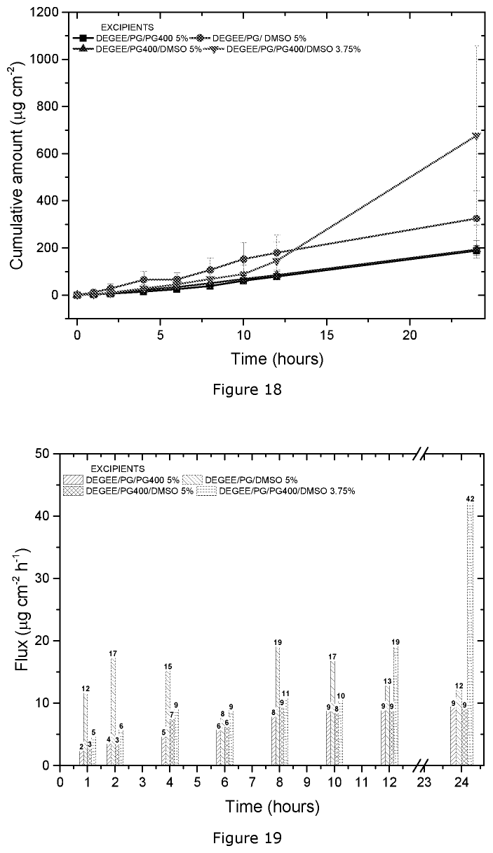

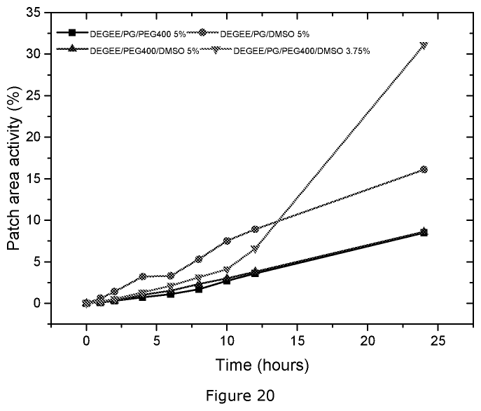

Combing the results of the previous part it can be concluded that the 2-(2-ethoxyethoxy) ethanol/DMSO (5 wt %), 2-(2-ethoxyethoxy) ethanol/PG (5 wt %) formulations are the most efficient and the addition of fatty compounds does not enhance the permeation of lidocaine through the tested membrane. We were interested to explore if the mixture of three or more excipients (maximum total amount 15 wt %), that always contain the combination of the efficient formulations, would improve the permeation any further. As it can be seen in FIGS. 18-20 this was the case only when all the excipients (3.75 wt % each) were employed and even then, the total permeated amount was not as high as in the 2-(2-ethoxyethoxy) ethanol/DMSO case.

### CONCLUSION

According to our study, 2-(2-ethoxyethoxy) ethanol/DMSO (5 wt % each) is the most efficient formulations which enhance the flux of lidocaine across Strat-M membrane.

1. A first composition for transdermal drug delivery comprising:
a drug for transdermal drug delivery;
a first excipient, the first excipient comprising alkoxy alcohol and a glycol.

2. A composition according to claim 1, wherein the drug is hydrophobic.

3. A composition according to claim 2, wherein the drug is ibuprofen.

4. A composition according to claim 1, wherein the drug is hydrophilic.

5. A composition according to claim 4, wherein the drug is lidocaine.

6. A composition according to any preceding claim, wherein the alkoxy alcohol is in the range C2-C10.

7. A composition according to claim 6, wherein the alkoxy alcohol is transcutol (2-(2-ethoxyethoxy)ethanol).

8. A composition according to any preceding claim, wherein the glycol is a C2-C6 glycol.

9. A composition according to claim 8, wherein the glycol is propylene glycol.

10. A composition according to any preceding claim, wherein the alkoxy alcohol is present in the range 1% to 20% by weight of the composition.

11. A composition according to claim 10, wherein the alkoxy alcohol is present in the range 2% to 10% by weight of the composition.

12. A composition according to any preceding claim, wherein the glycol is present in the range 1% to 20% by weight of the composition.

13. A composition according to claim 12, wherein the glycol is present in the range 2% to 10% by weight of the composition.

14. A composition according to any preceding claim, wherein the ratio of alkoxy alcohol to glycol is in the range 1:10 to 10:1.

15. A composition according to claim 14, wherein the ratio of alkoxy alcohol to glycol is in the range 1:2 to 2:1.

16. A composition according to any preceding claim, further comprising a preservative.

17. A composition according to claim 16, wherein the preservative is an aryl alcohol.

18. A composition according to any preceding claim, wherein the composition further comprises a fatty acid, fatty alcohol or fatty ester.

19. A composition according to claim 18, wherein the composition further comprises a fatty alcohol or fatty ester.

20. A composition according to claim 19, wherein the fatty alcohol is selected from octadecanol and/or wherein the fatty ester is isopropyl myristate.

21. A composition according to any of claims 18 to 20, wherein the fatty acid, fatty alcohol or fatty ester are present in the range 1 to 10% by weight of the composition.

22. A composition according to claim 21, wherein the fatty acid, fatty alcohol or fatty ester are present in the range 1 to 2% by weight of the composition.

23. Use of the first excipient according to any preceding claim as permeability enhancer for transdermal drug delivery.

24. Use according to claim 23, wherein the drug for transdermal delivery is hydrophobic.

25. Use according to claim 24, wherein the drug for transdermal delivery is ibuprofen.

26. Use according to claim 23, wherein the drug for transdermal delivery is hydrophilic.

27. Use according to claim 26, wherein the drug for transdermal delivery is lidocaine.

28. A second composition for transdermal drug delivery comprising:
a drug for transdermal drug delivery;
a second excipient, the second excipient comprising an alkoxy alcohol and a sulfoxide.

29. A composition according to claim 28, wherein the drug is hydrophilic.

30. A composition according to claim 29, wherein the drug is lidocaine.

31. A composition according to any of claims 28 to 30, wherein the alkoxy alcohol is in the range C2-C10.

32. A composition according to claim 31, wherein the alkoxy alcohol is transcutol (2-(2-ethoxyethoxy)ethanol).

33. A composition according to claim 32, wherein the alkoxy alcohol is present in the range 2% to 10% by weight of the composition.

34. A composition according to any of claims 28 to 33, wherein the sulfoxide is DMSO.

35. A composition according to any of claims 28 to 34, wherein the ratio of alkoxy alcohol to sulfoxide is in the range 1:10 to 10:1.

36. A composition according to claim 35, wherein the ratio of alkoxy alcohol to DMSO is in the range 1:2 to 2:1.

37. A composition according to any of claims 28 to 36, further comprising a preservative.

38. A composition according to claim 37, wherein the preservative is an aryl alcohol.

39. A composition according to any of claims 28 to 38, wherein the composition further comprises a fatty acid, fatty alcohol or fatty ester.

40. A composition according to claim 39, wherein the composition further comprises a fatty alcohol or fatty ester.

41. A composition according to claim 40, wherein the fatty alcohol is selected from octadecanol and/or wherein the fatty ester is isopropyl myristate.

42. A composition according to any of claims 39 to 41, wherein the fatty acid, fatty alcohol or fatty ester are present in the range 1 to 10% by weight of the composition.

43. A composition according to claim 42, wherein the fatty acid, fatty alcohol or fatty ester are present in the range 1 to 2% by weight of the composition.

44. A transdermal drug delivery patch comprising the first composition according to any of claims 1 to 22 or the second composition according to any of claims 28 to 43.

45. A patch according to claim 44, wherein the patch comprises a crossed-linked silyl containing polymer.

46. A patch according to claim 45, wherein the crossed-linked silyl containing polymer is selected from: silyl-containing polyethers, silyl-containing polyurethanes, silyl-containing polyesters, co-polymers thereof and/or combinations thereof.

47. A patch according to claim 46, wherein the co-polymers are block copolymers, random copolymers, alternating copolymers, graft copolymers or combinations thereof.

48. A patch according to any of claims 44 to 47, further comprising a tackifying resin.

49. Use of the second excipient according to any of claims 28 to 43 as permeability enhancer for transdermal drug delivery.

50. Use according to claim 49, wherein the drug for transdermal delivery is hydrophilic.

51. Use according to claim 50, wherein the drug for transdermal delivery is lidocaine.
